(19) United States
(12) Patent Application Publication
(10) Pub. No.: US 2025/0085280 A1
(43) Pub. Date: Mar. 13, 2025

(54) Methods for Detecting Antibody Self-Association

(71) Applicant: AMGEN INC., Thousand Oaks, CA (US)

(72) Inventors: Yuqing Shen, Thousand Oaks, CA (US); Emma Montserrat Pelegri-O'Day, Thousand Oaks, CA (US)

(21) Appl. No.: 18/730,373

(22) PCT Filed: Jan. 23, 2023
(86) PCT No.: PCT/US23/11321

§ 371 Date: Jul. 19, 2024

Related U.S. Application Data
(60) Provisional application No. 63/306,842, filed on Feb. 4, 2022.
(60) Provisional application No. 63/302,341, filed on Jan. 24, 2022.

(51) Int. Cl. G01N 33/564; G01N 33/543; G01N 33/553; G01N 33/68

(52) U.S. Cl. CPC G01N 33/564; G01N 33/54386; G01N 33/553; G01N 33/686

(57) ABSTRACT

The disclosure provides for methods and systems for detecting antibody protein product self-association comprising inducing co-agglutination of nanoparticles and solid supports to capture the nanoparticles, each coated with a ligand specific for immunoglobulins, and utilizing a fluidic device to detect co-agglutination and antibody protein product self-association.

This application claims priority to U.S. provisional application No. 63/302,341 filed Jan. 24, 2022 and U.S. provisional application No. 63/306,842 filed Feb. 4, 2022, both of which are incorporated herein by reference in their entirety.

### FIELD

The disclosure provides for methods and systems for detecting protein self-association comprising inducing co-agglutination of nanoparticles and solid supports to capture the nanoparticles, each coated with a ligand specific for immunoglobulins or receptors, and utilizing a fluidic device to detect co-agglutination and antibody protein product self-association.

### BACKGROUND

During the development of therapeutic antibodies, much time and resources are invested in the identification of potentially problematic physicochemical properties of antibodies to mitigate the risk for costly late-stage failures in the clinic. Early assessment of antibody attributes, especially prior to purification is an efficient and resource saving approach to lead molecule optimization process.

Fluidic systems may be used for the manipulation and analysis of samples comprising proteins such as antibodies. Optofluidic systems provide for high throughput single cell screening capability based on nanofluidic and opto-electronic positioning technology. This technology is based on light-induced electrokinetics that gives rise to designated forces on both solid and fluidic structures (Jorgolli et al., Biotechnol Bioeng 2019, 116(9), 2393-2411). For example, commercially available fluidic devices, such as the integrated technology of the Berkeley Lights (BLI) Beacon® Optofluidic System (Emeryville, CA) have the flexibility and capability for a broad array of applications applicable to commercial large molecule drug development, including antibody discovery, clonal selection, gene editing, linking phenotype to genotype, and cell line development.

### SUMMARY

There is a need to develop “on-microchip” assays predictive of molecule performance in shake flasks and bioreactors. Disclosed herein are methods to detect and characterize proteins, for example antibody protein products such as human or humanized antibodies with a propensity to self-associate. The methods may be performed in a fluidic device such as a chip of the BLI Beacon Optofluidic system, utilizing solid supports, such as micrometer-sized beads, and nanoparticles both coated with a ligand specific for an antibody protein product, such as anti-human IgG (huIgG) antibodies. Methods of detecting antibodies with a propensity to self-associate relate to solubility, aggregation, and viscosity, an important attribute for developability assessment.

Methods and systems for detecting antibody protein product association and aggregation are described. It is contemplated that co-agglutination of nanoparticles and a solid support, such as capture beads, causes a detectable change in optical signal. Examples of suitable optical signals include light emission (such as fluorescence emission), optical pattern, and light scattering (such as dynamic light scattering). First, antibody protein product aggregates may be incubated with nanoparticles that bind to the antibody protein product, and then with capture beads that further bind to the antibody protein product, causing co-agglutination of the nanoparticles and capture beads. The co-agglutination results in a change in absorbance so that the aggregates may be detected, for example by detecting a change in absorbance or in light emission. Additionally, the capture beads causing agglutination advantageously permit visualization of aggregates without a need to measure changes in absorbance or light emission. The process may be performed in a fluidic device, and agglutination may be detected in situ.

In some aspects, nanometer-scale anti-Fc coated nanoparticles are mixed with a sample of antibody protein products. If the antibody protein products self-associate, the nanoparticles form clusters. Micrometer-scale anti-Fc capture beads are then added. If the antibody protein products have self-associated, the capture beads co-agglutinate with the clusters of nanoparticles. The presence of the co-agglutinated structures can be detected as a change in optical pattern, or as a change in absorbance or light emission. This assay can be performed in situ in a fluidic device, such as in a BLI pen. This assay can be used for selecting clones that have a low risk of producing antibodies at risk of self-association.

For example in the disclosed methods, cells expressing antibody protein product are seeded into an individual pen or channel on a fluidic device. In some embodiments, the individual pen is on a microfluidic chip containing thousands of pens in the Beacon® system, through a light-induced electric field that positions each cell into an empty pen. Then antibody protein products secreted by the cell in each pen are analyzed for their properties with reagents imported onto the chip, and images acquired through brightfield or filter cubes by a charged coupled device (CCD) camera. The method may further comprise quantification of light emission of the capture beads, which allows for the identification of cells expressing antibody protein products with high risk of self-association, an attribute related to solubility, aggregation, and viscosity.

The disclosure provides for methods and systems of detecting antibody protein product association comprising a) contacting a sample comprising an antibody protein product with nanoparticles coated with a ligand for an antibody protein product, wherein the contacting occurs in a fluidic device under conditions that allow the antibody protein product to self-associate and thereby forming clusters of nanoparticles, b) contacting the clusters of nanoparticles with a solid support, wherein the solid support is coated with a ligand for the antibody protein product of (a), and c) detecting a change in optical signal of the nanoparticles and/or solid support, wherein a change in optical signal indicates antibody protein product association. Examples of optical signals include changes in light emission (such as changes in fluorescence emission), changes in light scattering (e.g., dynamic light scattering), and changes in optical pattern. For example, a change in absorbance may be indirectly observed by measuring light emission. Agglutination of metal nanoparticles such as gold nanoparticles may cause a change in absorbance, and thus metal nanoparticles may be useful for detection of changes in light emission. In some embodiments, the optical signal is detected using an emission filter. In some embodiments, the optical signal is detected using a fluorescence scan, for example the change in optical signal is detected using fluorescence scan wherein the solid support has an average of 6.5-10 μm, such as about 6.5 μm.

Changes in light scattering such as dynamic light scattering may be observed for agglutination of nanoparticles of metal and non-metal materials, and as such, metal (e.g., gold) as well as non-metal (e.g., polymer such as polystyrene) nanoparticles may be useful for detection of changes in light scattering (e.g., dynamic light scattering). Changes in optical pattern may be observed for agglutination of nanoparticles of any material. Microscopy may be used to detect changes in optical pattern. It is contemplated that machine learning may be useful in high-throughput identification of changes in optical pattern, for example on a fluidic device comprising hundreds or thousands of pens. A machine learning algorithm may be trained with images of agglutinated and/or non-agglutinated nanoparticles and solid supports, which may be used identify optical patterns that are indicative of agglutination.

In some embodiments, the disclosed methods and systems are used for the identification of cells expressing an antibody protein product, such as a monoclonal antibody or fragment thereof, that have a high risk of self-association. In additional embodiments, the disclosed methods and systems are used to select clones that are less likely to express an antibody protein product, such as a monoclonal antibody or fragment thereof, that has a high risk of self-association. In some embodiments, the sample comprises or consists of conditioned media.

In any of the disclosed methods, the antibody protein product comprises or consists of a large peptide (e.g., a peptide comprising at least 100 amino acid residues), antibody, antibody fragment, antibody fusion peptide or antigen-binding fragment thereof. In an exemplary embodiment, the antibody protein product is a human or humanized monoclonal antibody.

In some embodiments, the disclosed methods and systems are carried out with nanoparticles that comprise or consist of a metal or a polymer. Examples of metals for the nanoparticles include gold, silver, and palladium. Examples of polymers for the nanoparticles include polystyrene, poly(lactide-co-glycolide) (PLGA), polyethylene oxide (PEO), polyethylene glycol (PEG), and polyvinyl alcohol (PVA). Additionally, the nanoparticles may include silica glass, quartz, chitosan, dextran, alginate, gadolinium, or carbon. It is contemplated that metal nanoparticles can be used to detect changes in absorbance and/or light emission, and that nanoparticles of any material described herein can be used to detect changes in light scattering (such as dynamic light scattering) or optical pattern. Changes in agglutination of metal can cause changes in absorbance, which can be detected as changes in light emission. Changes in optical patterns can be detected by microscopy. As some metal nanoparticles such as silver and palladium may disperse in solution, electron microscopy may be used to detect changes in optical patterns of such metal nanoparticles. Without being limited by theory, it is contemplated that silver is negatively charged, and is not amenable to being passively coated with protein ligands such as antibodies. Accordingly, gold nanoparticles may have advantages over silver nanoparticles for coating the nanoparticle with ligand.

In some embodiments, the disclosed methods and systems are carried out with nanoparticles having a mean diameter ranging in size between about 1 nm to about 100 nm, or about 10 nm to about 50 nm, or about 20 nm to about 60 nm, or about 30 nm to about 70 nm, or about 40 nm to about 80 nm, or about 50 nm to about 100 nm, or about 10 nm to about 40 nm, or about 10 to about 30 nm, or about 20 nm to about 50 nm, or about 30 nm to about 50 nm. In exemplary embodiments, the disclosed methods are carried out with nanoparticles that are about 10 nm, or about 20 nm, or about 30 nm, or about 40 nm, or about 50 nm, or about 60 nm, or about 70 nm, or about 80 nm, or about 90 nm, or about 100 nm.

As used herein, the “ligand” for an antibody protein product refers an agent that binds to the antibody protein product such as an antibody that binds the antibody protein product or a binding partner for the antibody protein product. Examples of ligand for purposes herein include an antigen for the antibody protein product, an anti-idiotype antibody, anti-Fc antibody, protein A, or protein G. In exemplary embodiments, the solid support (e.g., bead) comprises anti-FC protein, protein A or protein G, or the solid support (e.g., bead) comprises protein A or protein G. In some embodiments, the disclosed methods are carried out with nanoparticles coated with a ligand for an antibody, wherein the ligand is an antibody or fragment thereof that specifically binds human Fc protein, or an antibody or fragment thereof that binds to a human immunoglobulin such as anti-human IgG, anti-human IgM, anti-human IgA, or anti-human IgD or anti-human IgE. In other embodiments, the nanoparticles are coated with a ligand for an antibody, wherein the ligand is protein A or protein G, or a combination thereof. The term “coated” refers to attaching or covalently coupling the ligand to the nanoparticle, including, by way of example, passive absorption.

In various embodiments, the disclosed methods and systems are carried out with a solid support that is a bead or a microsphere, a membrane, nanofiber, nanotube, resin or agarose. For example, the solid support may be polymer-based e.g. polystyrene beads, poly(lactide-co-glycolide) (PLGA) beads, polyethylene oxide (PEO) beads, polyethylene glycol (PEG) beads, polyvinyl alcohol (PVA) beads, or may be metal-based e.g. gold beads. In addition, the solid support may be chitosan, dextran, alginate, gadolinium-based, carbon-based, silica-based or iron-based. When the solid support is a resin, the resin may be a polymeric resin such as cellulose, polystyrene, agarose, polyacrylamide or agarose. In some methods, the beads may be magnetic beads.

For example, the solid support may comprise or consist of beads having a mean diameter of at least about 1 μm, 2 μm, 3 μm, 4 μm, 5 μm, 6 μm, 6.5 μm, 7 μm, 8 μm, 9 μm, 10 μm, 20 μm, 30 μm, 40 μm or 50 μm, including ranges between any two of the listed values, for example about 1-5 μm, about 1-10 μm, about 1-20 μm, about 1-40 μm, about 5-10 μm, about 5-20 μm, about 5-40 μm, about 6.5-10 μm, about 10-20 μm, or about 10-40 μm. Without being limited by theory, it is contemplated that solid support having a greater diameter than the nanoparticle has a greater antibody protein product binding capacity than each nanoparticle, which can facilitate co-agglutination. By way of example, the solid support may have a mean diameter at least 2×, 5×, 10×, 50×, 100×, 500× or 1000× that of the nanoparticle, such as 2×-100×, 2×-500×, 2×-1000×, 10×-100×, 10×-500×, 10×-1000×, 100×-500×, or 100×-1000× that of the nanoparticle. The diameter may be determined as a mean diameter for a population of solid supports or nanoparticles.

In any of the disclosed methods, the solid support is coated with an antibody or a fragment thereof that specifically binds an antibody protein product, such as anti-human IgG, anti-human IgM, anti-human IgA, or anti-human IgD or anti-human IgE. In other embodiments, the solid support is coated with a ligand for an antibody protein product, wherein the ligand is protein A or protein G, or a combination thereof. The term “coated” refers to attaching or covalently coupling the ligand to the nanoparticle.

In some embodiments, the solid support comprises a unique barcode. Examples of suitable barcodes include peptides or nucleic acids having unique sequences or molecular weights, pigments or combinations of pigments, glycans and/or carbohydrates or combinations thereof or fluorophores or combinations of fluorophores.

In any of the disclosed methods, the sample is any liquid or formulation comprising an antibody, antibody protein product or a fragment thereof. In various embodiments, the sample is a fluid comprising an antibody, antibody protein product or fragment thereof that is to be processed, measured or analyzed for stability and/or structural integrity or other attributes. In some embodiments, the sample comprises or consists of conditioned media or any liquid from which an antibody, antibody protein product or fragment thereof is purified or isolated. In some embodiments, the sample comprises a cell which expresses the antibody, antibody protein product or fragment thereof.

In any of the disclosed methods, the sample comprises an antibody protein product, such as an antibody, antibody protein product, bispecific T-cell engager (BiTER) molecule, antibody fragment, antibody fusion peptide or antigen-binding fragment thereof, or peptide. In related embodiments, the antibody is a polyclonal or monoclonal antibody. As used herein, the term “antibody protein product” refers to antibodies, as well as any one of several antibody alternatives which in various instances is based on the architecture of an antibody but is not found in nature. An “antibody” is a subgenus of antibody protein product. It refers to refers to an immunoglobulin of any isotype with specific binding to the target antigen, and includes, for instance, monoclonal antibodies. Antibodies may be of any suitable host species, for example, chimeric, humanized, fully human, fully mouse, fully rabbit, or fully llama. An antibody generally comprises two full-length heavy chains and two full-length light chains. For example, human antibodies can be of any isotype, including IgG (including IgG1, IgG2, IgG3 and IgG4 subtypes), IgA (including IgA1 and IgA2 subtypes), IgM and IgE. In some aspects, the antibody protein product has a molecular-weight within the range of at least about 12 kDa-10 MDa, for example at least about 12 kDa-5 MDa, 12 kDa-1 MDa, 12 kDa-750 KDa, at least about 12 kDa-250 kDa, or at least about 12 kDa-150 kDa. In certain aspects, the antibody protein product has a valency (n) range from monomeric (n=1), to dimeric (n=2), to trimeric (n=3), to tetrameric (n=4), if not higher order valency. Antibody protein products in some aspects are those based on the full antibody structure and/or those that mimic antibody fragments which retain full antigen-binding capacity, e.g., scFvs, Fabs and VHH/VH (discussed below). The smallest antigen binding antibody fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats comprising scFvs linked to oligomerization domains. The smallest fragments are VHH/VH of camelid heavy chain Abs as well as single domain Abs (sdAb) including UniDab® construct-containing molecules and UniAb® constructs (TeneoBio). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody comprises a biologically active peptide grafted onto an Fc domain. Peptibodies are well-described in the art. See, e.g., Shimamoto et al., mAbs 4 (5): 586-591 (2012). Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015). In exemplary aspects, the antibody protein product comprises or consists of a bispecific T cell engager (BiTER) molecule, which is an artificial bispecific monoclonal antibody. BiTER molecules are fusion proteins comprising two scFvs of different antibodies. One binds to CD3 and the other binds to a target antigen. BiTE® molecules are known in the art. See, e.g., Huehls et al., Immuno Cell Biol 93 (3): 290-296 (2015); Rossi et al., MAbs 6(2): 381-91 (2014); Ross et al., PLOS One 12(8): e0183390.

In any of the disclosed methods, the method can be carried out with any fluidic system, fluidic device or fluidic apparatus known in the art, for example an optofluidic device. A fluidic device (or fluidic apparatus) is a device that includes one or more discrete circuits configured to hold a fluid, each circuit comprised of fluidically interconnected circuit elements. The circuit element including but not limited to region(s), flow path(s), channel(s), chamber(s), and/or pen(s), and at least one port configured to allow the fluid to flow into and/or out of the fluidic device. The fluidic circuit may be configured to have a first end fluidically connected with a first port (e.g., an inlet) in the fluidic device and a second end fluidically connected with a second port (e.g., an outlet) in the fluidic device or connected to a second fluidic device or a second region, flow path, channel, chamber or pen in the fluidic device. The fluidic device may be a microfluidic device, through other scales such as nano-scale may also be suitable. For example, the fluidic device may be a microfluidic chip, microfluidic channel, microfluidic cell, nanofluidic chip, nanofluidic channel, nanofluidic cell or sequestration pen. In some embodiments, the fluidic system comprises a multi-well plate, for example a 96- or 384-will plate. The multi-well plate may be in fluid communication with a circuit.

For a microfluidic device, the circuit will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of fluidic device having a fluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements is configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A “fluidic channel” or “flow channel” as used herein refers to a flow region of a fluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is in the range of from about 50,000 microns to about 500,000 microns, including any range there between. In some embodiments, the horizontal dimension is in the range of from about 100 microns to about 1000 microns (e.g., about 150 to about 500 microns) and the vertical dimension is in the range of from about 25 microns to about 200 microns, e.g., from about 40 to about 150 microns. It is noted that a flow channel may have a variety of different spatial configurations in a fluidic device, and thus is not restricted to a perfectly linear element. For example, a flow channel may include one or more sections having any of the following configurations: curve, bend, spiral, incline, decline, fork (e.g., multiple different flow paths), and any combination thereof. In addition, a flow channel may have different cross-sectional areas along its path, widening and constricting to provide a desired fluid flow therein.

In some embodiments, systems are described. The system may comprise a fluidic device. The system may further comprise nanoparticles coated with a ligand for an antibody protein product. The system may further comprise solid supports coated with a ligand for the antibody protein product. For some systems, a mean diameter of the solid supports is greater than that of the nanoparticles. The system may further comprise an optical detector configured to detect changes in optical signal, e.g. change in absorbance or light emission, of the solid supports.

### BRIEF DESCRIPTION OF THE DRAWINGS

FIG. 1 is a schematic illustrating the capture beads and nanoparticles co-agglutination assay.

FIG. 2 provides brightfield OEP and filter TRed images showing there was no co-agglutination pattern detected nor any emission signal detected when the anti-huFc coated gold nanoparticles were mixed with capture beads coated with anti-huIgG in the absence of an added antibody.

FIG. 3 provides brightfield OEP and filter TRed images after mixing mAb1 (negative control) with anti-huFc coated gold nanoparticles and 6.5 μM capture beads coated with anti-huIgG. No bead co-agglutination or emission signal were detected in the presence of 1 μM of mAb1.

FIG. 4 provides brightfield OEP and filter TRed images after mixing mAb2 (positive control) with anti-huFc coated gold nanoparticles and 6.5 μM capture beads coated with anti-huIgG. Islands of co-agglutination of anti-huIgG capture beads and anti-huFc gold nanoparticles were detected with increased emission signals in the presence of 1 μM mAb 2.

FIG. 5 is brightfield OEP images showing that anti-huFc coated gold nanoparticles (left panel) generated more visible specks compared to uncoated gold nanoparticles (right panel), after a 1 hour incubation with the positive control antibody, mAb 2. This was carried out in the absence of capture beads.

FIG. 6 is brightfield OEP and filter TRed images showing there was no co-agglutination pattern detected nor any emission signal detected when the anti-huFc coated gold nanoparticles were mixed with 3 μm capture beads coated with anti-huIgG in the presence of CHOK1 growth media but in the absence of an added antibody.

FIG. 7 provides brightfield OEP and filter TRed images after mixing mAb1 (negative control) in CHO-K1 media with anti-huFc coated gold nanoparticles and 3 μM capture beads coated with anti-huIgG. No bead co-agglutination or emission signal were detected in the presence of 1 μM of mAb1.

FIG. 8 provides brightfield OEP and filter TRed images after mixing mAb2 (positive control) in CHO-K1 media with anti-huFc coated gold nanoparticles and 3 μM capture beads coated with anti-huIgG. Islands of co-agglutination of anti-huIgG capture beads and anti-huFc gold nanoparticles were detected with strong emission signals in the presence of 1 μM mAb 2.

FIG. 9 provides brightfield OEP and filter TRed images taken from Beacon® pens comprising live cultures of mAb-expressing CHO-K1 cells which were mixed with anti-huFc coated 40 nm gold nanoparticles and 3 μM capture beads coated with anti-huIgG prior to imaging.

FIGS. 10A-10C demonstrate increased peak fluorescence emission detected for mAb2 (positive control) and anti-huFc gold nanoparticles mixture in the presence of capture beads compared with mAb1 (negative control). FIG. 10A demonstrates that 10 μm capture beads generated separation of peak fluorescence emission between mAb2 and mAb1. FIG. 10B demonstrates that 6.5 μm capture beads generated the biggest separation of peak fluorescence emission between mAb2 and mAb1.

### DETAILED DESCRIPTION

The disclosed methods utilize nanoparticles coated with a ligand for an antibody protein product, such as an anti-human immunoglobulin, e.g., anti-huIgG antibodies, in affinity-capture self-association nanoparticle spectroscopy for the detection of antibody protein product self-association at a low concentration. Those antibody protein products which tend to self-associate cause the nanoparticles to cluster, resulting in a change of optical signal, e.g. a change in absorbance or light emission. Due to lack of optical signal measurement on some fluidic systems, another form of signal detection may be used. Solid supports also coated with a ligand for an antibody protein product, such as an anti-human immunoglobulin (e.g. anti-huIgG) coated polystyrene beads may be used to capture the clusters of nanoparticles and amplify the signal through co-agglutination of the nanoparticles on the surface of the solid support. The amplified signal may be detected through emission filters on an optofluidic device such as the Beacon® system. In some methods, changes in patterns of solid supports may be detected by microscopy. Detecting the changes in patterns may further comprise image processing. The image processing may be automated, and may comprise machine learning.

As cells in an individual pen of the chip on the Beacon® system express antibodies continuously, in exemplary disclosed methods the secreted antibody are captured by imported nanoparticles diffused in solution. The presence of self-associating antibodies causes the particles to form clusters and the addition of micrometer-sized capture solid supports (e.g. capture beads) into each pen, cause the clusters of nanoparticles to co-agglutinate onto the solid support. The co-agglutination on the solid support results in a change in the solid support pattern and light emission captured by a CCD camera. Thus, quantification of emission signal intensity allows for the identification of cells expressing antibodies with high risk of self-association. Also, modification of filter cube excitation and emission wavelength is considered to improve signal-to-noise ratio for detection at the optimal wavelength.

In some embodiments, the solid support is a capture bead having a large antibody binding capacity of 1e5 molecule/bead, while only about 170 antibody molecules per nanoparticle, thus the inclusion of the capture solid support allows for detecting antibodies at a low concentration. When particle-bound antibody has a strong tendency of self-association, it results in particles aggregating and absorbance (and light emission) changing. The ability for the nanoparticles to pack at high density due to their small size is an advantage for detection. Co-agglutination is used for image-based detection with beads pattern change and absorbance or light emission measurement for quantification.

The fluidic device allows for growing and expanding a single cell within a chamber or sequestration pen, which in turn allow for clonal selection of the cell producing the antibody protein product to be detected. The clonal selection allows for selection of the clones for large-scale protein production and purification during drug discovery and biologic drug manufacturing, e.g. antibody production. The disclosed methods also allow for continual analysis of the cells as they are expanding, and the assays can be repeated on the same growing cell.

A colony of biological cells is “clonal” if all of the living cells in the colony that are capable of reproducing are daughter cells derived from a single progenitor cell. In certain embodiments, all the daughter cells in a clonal colony are derived from the single progenitor cell by no more than 10 divisions. In other embodiments, all the daughter cells in a clonal colony are derived from the single progenitor cell by no more than 14 divisions. In other embodiments, all the daughter cells in a clonal colony are derived from the single progenitor cell by no more than 17 divisions. In other embodiments, all the daughter cells in a clonal colony are derived from the single progenitor cell by no more than 20 divisions. The term “clonal cells” refers to cells of the same clonal colony.

As used herein, a “colony” of biological cells refers to 2 or more cells (e.g. about 2 to about 20, about 4 to about 40, about 6 to about 60, about 8 to about 80, about 10 to about 100, about 20 about 200, about 40 about 400, about 60 about 600, about 80 about 800, about 100 about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

In some embodiments, the sample comprises or consists of conditioned media or any liquid from which the antibody, antibody protein product or fragment thereof may be purified or isolated. In various embodiments, the sample so subjected to the methods disclosed herein, comprises or consists of a large peptide, antibody, antibody fragment, antibody fusion peptide or antigen-binding fragments thereof. In related embodiments, the antibody is a polyclonal or monoclonal antibody.

### Fluidic Devices

Fluidic devices refer to an apparatus that use small amounts of fluid to carry out various types of analysis. The fluidic device comprises one or more discrete circuits configured to hold a fluid, each circuit comprised of fluidically interconnected circuit elements. The circuit element including but not limited to region(s), flow path(s), channel(s), chamber(s), and/or pen(s), and at least one port configured to allow the fluid to flow into and/or out of the fluidic device. These devices use chips, cells, channel, or sequestration pens that contain the fluid for analysis.

Fluidic devices such as microfluidic devices generally have one or more channels with at least one dimension less than 1 mm. Common fluids used in fluidic devices include whole blood samples, bacterial cell suspensions, protein or antibody solutions and various buffers. Fluidic devices can be used to obtain a variety of measurements including molecular diffusion coefficients, fluid viscosity, pH, chemical binding coefficients and enzyme reaction kinetics. Other applications for fluidic devices include capillary electrophoresis, isoelectric focusing, immunoassays, flow cytometry, sample injection of proteins for analysis via mass spectrometry, PCR amplification, DNA analysis, cell manipulation, cell separation, cell patterning and chemical gradient formation. Many of these applications have utility for clinical diagnostics.

The advantages for using fluidic devices include that the volume of fluids within these channels is very small, usually several nanoliters, and the amounts of reagents and analytes used is quite small. Moreover, when analyzing protein-producing cells, a relatively small number of cells (or even single cells) can produce a sufficient quantity and concentration of protein for analysis, reducing or avoiding incubation times for colony expansion. The fabrication techniques used to construct microfluidic devices are relatively inexpensive and are very amenable both to highly elaborate, multiplexed devices and also to mass production. Fluidic technologies enable the fabrication of highly integrated devices for performing several different functions on the same substrate chip.

Any fluidic device can be used (or modified to be used) in the disclosed methods, including commercially available devices. The fluidic device may be configured for use in an optofluidic system, which can use light to manipulate matter in the fluidic device such as cells. Described herein as an exemplary microfluidic device is a chip comprising the Berkley Lights (BLI) pen. For example, the BLI pen may be analyzed in The Beacon® Optofluidic System, the Lightning™ Optofluidic System or the Culture Station System (BLI. Emeryville, CA). Other exemplary optofluidic systems are the Cyto-Mine® System (Sphere Fluidics, Great Abington, Cambridge, UK).

### EXAMPLES

### Example 1

### General Methods

The nanoparticles and capture beads used in the experiments described herein were available from commercial sources as described Table 1. Purified antibodies were used as controls: an antibody (denoted as mAb2) known to demonstrate high self-association and viscosity score was used as a positive control; and an antibody (denoted as mAb1) known to demonstrate low self-association and viscosity score was used as a negative control. The attributes of the control antibodies are described in Table 2.

The tested control antibodies were stored at 4° C. prior to testing and were not subjected to any stress before or during the test. The control antibodies were premixed with the gold nanoparticles, and subsequently the capture beads added to the mixture. The mixture was then loaded on Beacon® system. PBS pH7.4 buffer and CHO-K1 growth media were used for background detection. Images were taken on the Beacon® system under Brightfield (OEP) and filter cube with Excitation 562/40 nm and Emission 624/40 nm (TRed 620 nm).

TABLE 1
Vendor information for coated gold
colloids and beads and the BLI Chip

| | |
|---|---|
| Anti-huFc_Gold nanoparticles 40 nm | Jackson ImmunoResearch Lab, |
| Unconjugated gold nanoparticles 20 nm | Ted Pell, Inc. |
| Anti-huIgG H + L coated beads 6.5 μm | Spherotech, Inc. |
| Anti-huFc coated beads 3 μm | Spherotech, Inc. |
| OptoSelect 3500 chip | Berkeley Lights, Inc. |

TABLE 2
Attributes of the control antibodies

| AC-SINS | AC-SINS | AC-SINS | Viscosity | | | | |
|---|---|---|---|---|---|---|---|
| Test Conc | λ max | Δ λ max | Conc | Viscosity | SEC Incubation | % HMW | Δ HMW |
| 1 mg/ml | (nm) | (nm) | (mg/mL) | (cP) | Conc (mg/mL) | T0 | 4 wk 40 C. |
| MAb 1 | 535.7 | 0 | 140 | 7.2 | 140 | 0.42 | 1.21 |
| MAb 2 | 561.7 | 26 | 157 | 25.6 | 70 | 0.63 | 2.16 |

AC-SINS: Affinity-Capture Self-Interaction Nanoparticle Spectroscopy

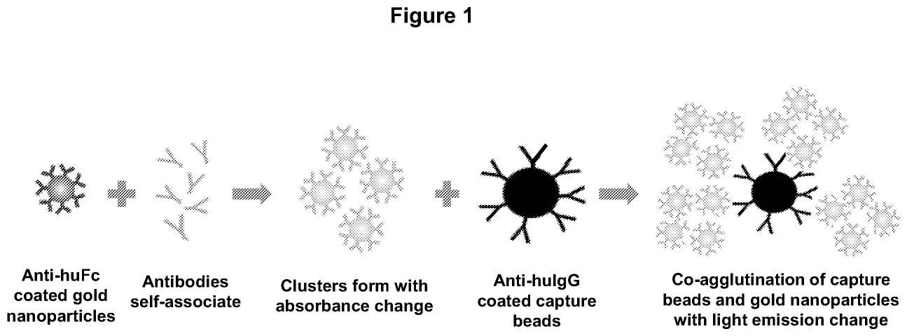

FIG. 1 summarizes the disclosed method and the experiments carried out in the following examples which detect antibody association and aggregation. Gold nanoparticles (40 nm) coated with polyclonal goat anti-human Fc fragments were used to bind mAbs of interest in a first incubation step (denoted as the “premix”). For associative mAbs, gold nanoparticles will cluster in the premix. Micrometer-scale anti-Fc solid supports (e.g., “capture beads”) that further bind to the mAb of interest are then added to the premix, causing co-agglutination of the gold nanoparticles and capture beads. The presence of the co-agglutinated structures caused a change in light absorbance and a change in the optical pattern of the mixture. Therefore, quantification of the absorbance change or image-based detection of changes in the optical pattern allowed for the identification of cells expressing antibodies with high risk of self-association, an attribute related to solubility, aggregation, and viscosity. In contrast, non-associative antibodies do not cause gold nanoparticles to cluster, or the capture beads to co-agglutinate and as a result there is no shift in light absorbance or optical patterns.

### Example 2

### Detection of Antibody Self-Association Using Gold Nanoparticles and Micrometer Beads

A combination of antibody-coated gold nanoparticles and antibody coated solid support, i.e. anti-IgG coated capture beads, was used to detect antibody self-association in an optofluidic device. PBS buffer (10 μl) or 1 μM purified antibody (10 μl mAb 1 or mAb2) was mixed with 40 nm gold nanoparticles coated with anti-huFc_(10 μl) in a tube for 15 minutes at room temperature (denoted as the “pre-mix). 120 μl of 6.5 μm anti-hu-IgG (heavy and light chain) coated beads (denoted as capture beads) were concentrated by centrifugation and then the capture beads were resuspended in 10 μl of the premix. The mixture was then loaded in the channel of chip 3500 on the Beacon® system. The images were recorded through Brightfield (OEP) and TRed filters. The assay was also carried out by mixing the premix in the absence of capture beads, which was also loaded in the channel of chip 3500 on the Beacon® system for imaging.

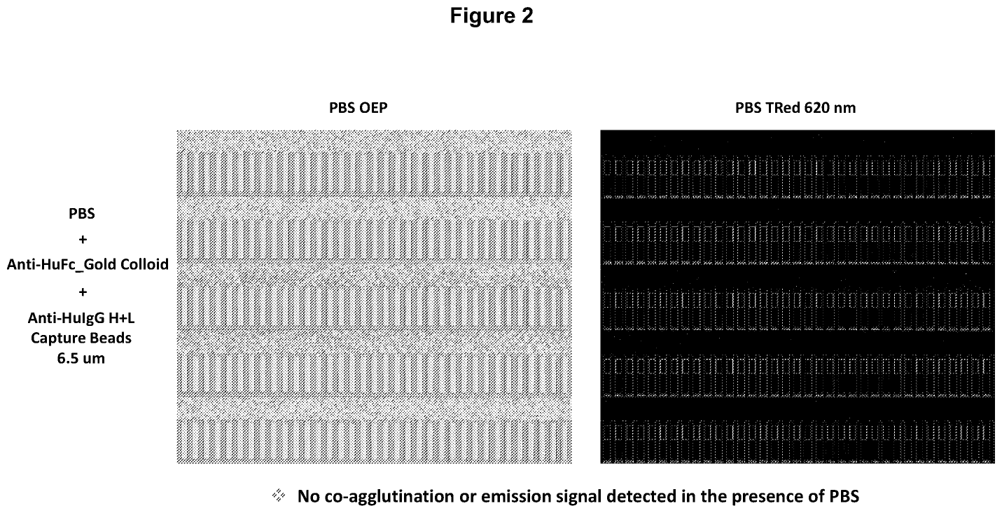

The pre-mix containing PBS buffer (without added antibody) was incubated with capture beads as described above. As shown in FIG. 2, there was no co-agglutination pattern of anti-huFc_gold nanoparticles mixed with capture beads as detected using the brightfield OEP. In addition, there was no emission signal detected through filter TRed.

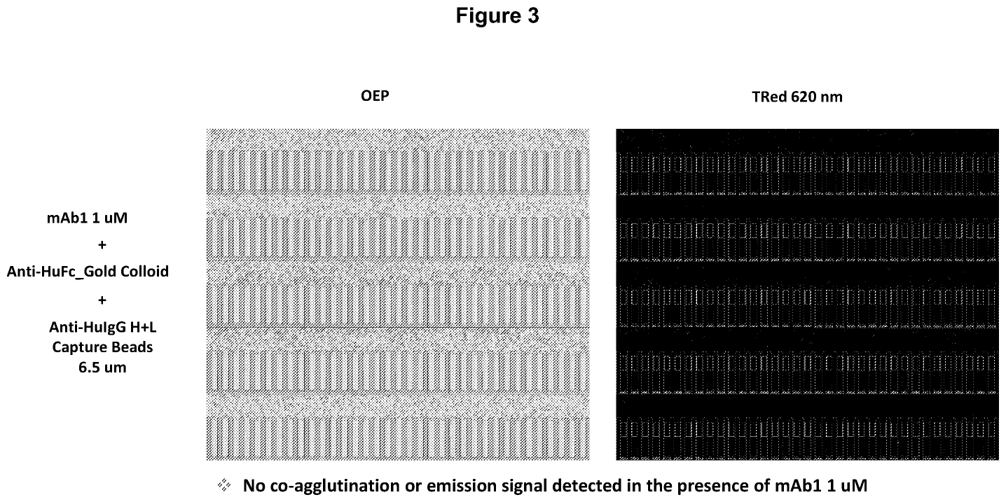

The premix containing 1 μM mAb 1 (negative control) was incubated with capture beads as described above. As shown in FIG. 3, no co-agglutination pattern of anti-FC gold nanoparticles mixed with the capture beads as detecting using the brightfield OEP. In addition, no emission signal were detected through the filter TRed.

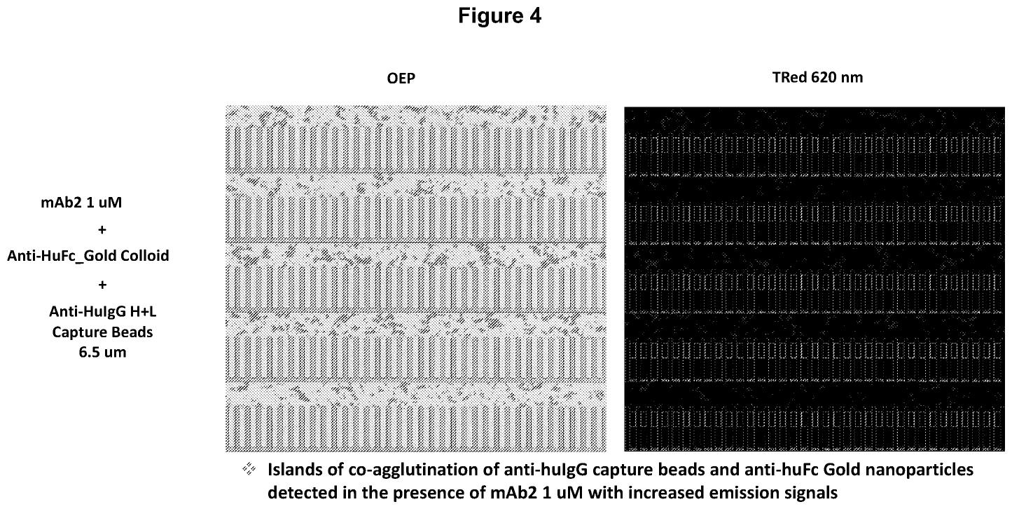

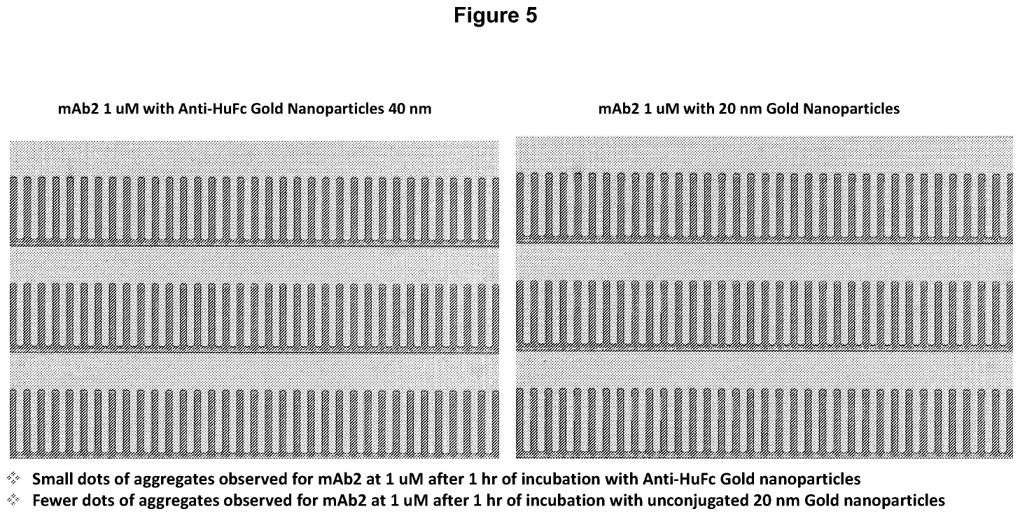

The premix containing 1 μM mAb 2 (positive control) was incubated with capture beads as described above. However, FIG. 4 shows the detection of a distinct co-agglutination pattern of gold nanoparticles with capture beads and demonstrated increased emission signals through filter TRed, in the presence of the positive control antibody (mAb 2). This set of results indicates that the approach of adding capture beads to the mix of antibody and gold nanoparticles was able to distinguish purified antibodies with high risk of self-association from those antibodies with a low risk of self-association on the Beacon® system. As shown in the brightfield images in FIG. 5, the premix containing 1 μM mAb 2 and 40 nm anti-huFc coated gold nanoparticles (left panel) without capture beads, generated more visible specks compared to unconjugated 20 nm gold nanoparticles (right panel). This experiment demonstrates that the change in brightfield OEP indicates that anti-hu-IgG coated nanoparticles specifically bound to mAb 2 with strong affinity while unconjugated gold nanoparticles rely on slow passive adsorption of antibody onto the positively charged gold. This experiment demonstrates that gold nanoparticles are able to form clusters in the presence of mAb 2.

### Example 3

Detection of Antibody Self-Association Using Gold Nanoparticles and Micrometer Beads in the Presence of Cell Culture Media

To determine whether the capture beads and gold nanoparticles co-agglutination assay and imaging could be carried out in the presence of culture media, a similar set of tests as those described in the preceding example were conducted using the same purified control antibodies and 3 μm anti-human Fc coated capture beads in the presence of fresh CHO-K1 culture media. Here, 1 μM purified antibody (5 μL) in CHO-K1 growth media (5 μL) or CHO-K1 growth media (5 μL) alone were mixed with 40 nm anti-huFc_Gold nanoparticles (5 μL) in a tube for 15 min at room temperature (denoted “premix”). 120 μl of 6.5 μm anti-hu-IgG (heavy and light chain) coated beads (denoted as capture beads) were concentrated by centrifugation and then the capture beads were resuspended in 10 μl of the premix. The mixture was loaded to the channel of chip 3500 on Beacon® system. The images were recorded through brightfield (OEP) and filter TRed.

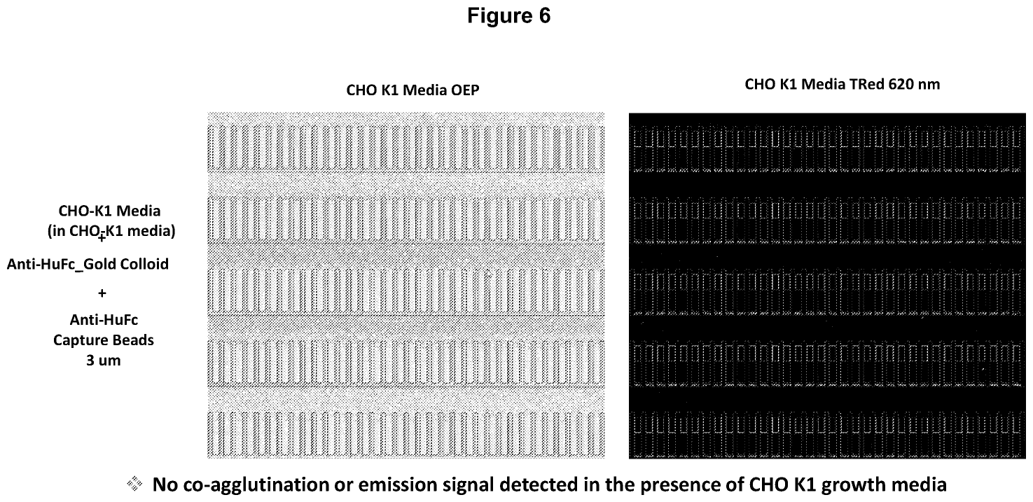

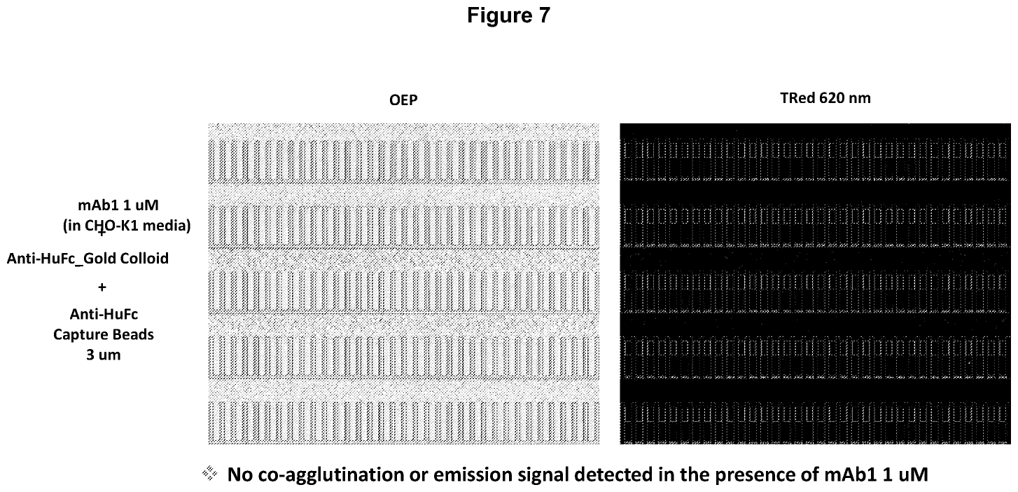

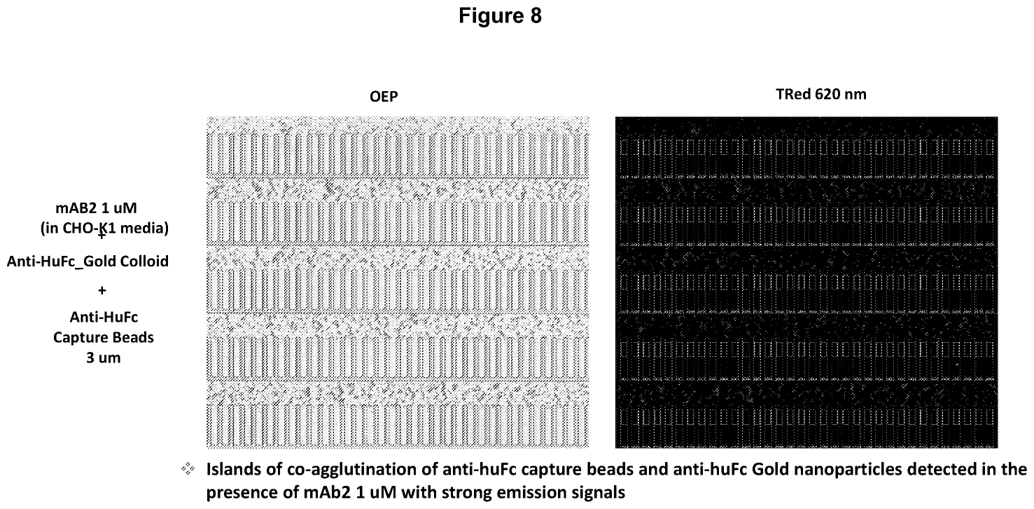

As shown in FIG. 6 and FIG. 7, no co-agglutination or emission signal was observed in the presence of fresh CHO-K1 culture media alone, or negative control antibody mAb 1 in the culture media, indicating that there was no non-specific binding interference by the culture media. The premix containing 1 μM mAb 2 (positive control) in the culture media was incubated with capture beads as described above. As shown in FIG. 8, there was a distinct co-agglutination pattern of gold nanoparticles mixed with 3 μm anti-huFc coated beads, and increased emission signals detected through filter TRed. These results further suggest that the capture beads and gold nanoparticles co-agglutination method could be used to assess antibody self-association propensity.

### Example 4

### Detection of Antibody Self-Association in Conditioned Media in a Optofluidic Device

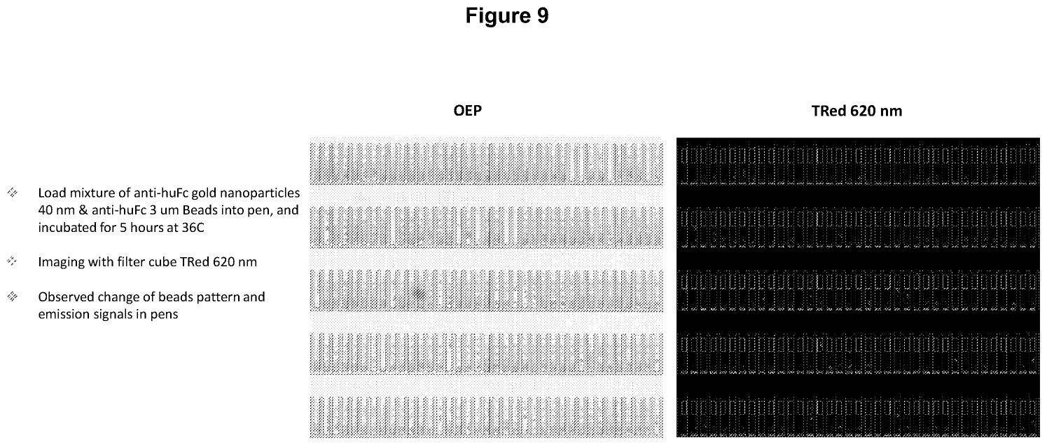

An experiment was conducted to test the co-agglutination assay using coated gold nanoparticles and capture beads in Beacon® pens comprising live CHO-K1 cells which express monoclonal antibodies. The cells were cultured on a fluidic chip for 48 hours in growth media. Subsequently, a premix of 40 nm anti-huFc_gold nanoparticles and 3 μm anti-huFc coated beads were loaded into the fluidic pens and incubated for 5 hours before imaging. Images were captured through brightfield and cube filter TRed 620 nm, 5 hours post loading. As shown in FIG. 9, a change of bead optical patterns and emission signals was observed in some pens. This experiment demonstrates at that the co-agglutination assay can be carried out on the conditioned media of living cells growing in an optofluidic device.

### Example 5

### Detection of Antibody Self-Association in Using A Fluorescent Scan

An experiment was carried to determine if a fluorescent scan can be used to detect antibody aggregates as detected using the methods described in the Examples above. A combination of antibody-coated gold nanoparticles and antibody-coated solid support, i.e. anti-IgG coated capture beads, was used to detect antibody self-association using a fluorescent scan. Equal volumes of 1 μM purified antibody (mAb1-negative control or mAb2-positive control) was mixed with 40 nm gold nanoparticles coated with anti-huFc in a tube for 15 minutes at room temperature (denoted as the “pre-mix”). 100 μl of 6.5 μm anti-hu-IgG (heavy and light chain) coated beads (denoted as capture beads) were concentrated by centrifugation and then the capture beads were resuspended in 10 μl of the premix. Aliquots of the pre-mix were added to equal volume of 5% capture beads in size of 10 μm, 6.5 μm, and 3 μm, respectively.

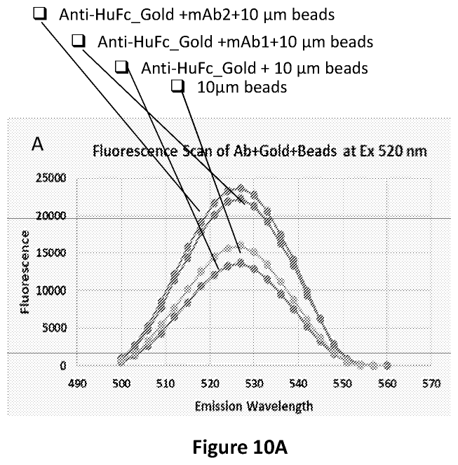

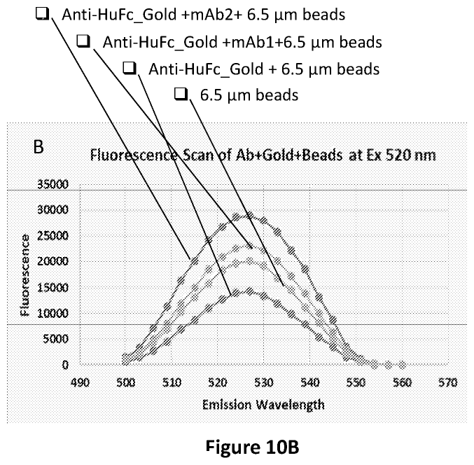

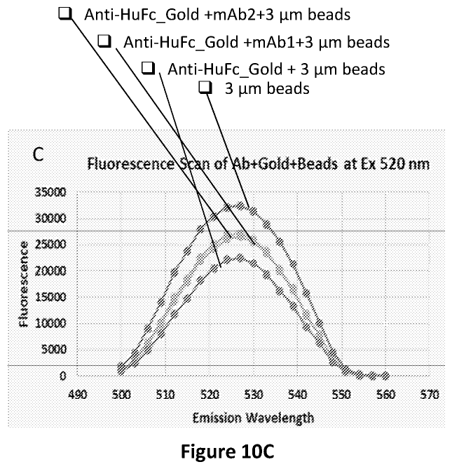

After 15 minutes incubation at room temperature, fluorescence scan was performed in black plate on Tecan SAFIRE II at an excitation wavelength of 520 nm. As shown in FIGS. 10A-C, increased peak fluorescence emission was detected for positive control mAb 2 compared with negative control mAb 1. At the excitation wavelengths of 520 nm, solid supports having a mean diameter of 6.5-10 μm showed greater separation than solid supports having a mean diameter of 3 μm. Capture beads in size of 6.5 μm generated biggest separation of peak fluorescence emission between mAb1 and mAb2 (see FIG. 10B).

The data demonstrate that a fluorescence scan can be used to detect agglutination. In particular, this experiment demonstrated that peak fluorescence emission could be used to discriminate antibodies with different self-association risks.

What is claimed is:

1. A method of detecting antibody protein product association comprising
a) contacting a sample comprising an antibody protein product with nanoparticles coated with a ligand for an antibody protein product, wherein the contacting occurs in a fluidic device under conditions that allow antibody protein products to self-associate, thereby forming clusters of nanoparticles,
b) contacting the clusters of nanoparticles with a solid support, wherein the solid support is coated with a ligand for the antibody protein product of (a), and
c) detecting a change in optical signal of the nanoparticles and/or the solid support, wherein a change in optical signal indicates antibody protein product association.

2. The method of claim 1 wherein the sample comprises conditioned media.

3. The method of any of the preceding claims, wherein the antibody protein product comprises or consists of a large peptide, antibody, antibody fragment, antibody fusion peptide or antigen-binding fragment thereof.

4. The method of any of the preceding claims, wherein the nanoparticles have a mean diameter ranging in size between about 10 nm to about 50 nm.

5. The method of any of the preceding claims, wherein the nanoparticles have a mean diameter of about 20 nm or about 30 nm.

6. The method of any of the preceding claims, wherein the nanoparticles are coated with an antibody or fragment thereof that specifically binds human Fc protein, protein A or protein G, or a combination thereof.

7. The method of any of the preceding claims, wherein the solid support is a bead, resin or agarose.

8. The method of any of the preceding claims, wherein the nanoparticle comprises or consists of a metal or a polymer.

9. The method of claim 8, wherein the nanoparticle comprises or consists of gold.

10. The method of any of the preceding claims, wherein the solid support is coated with an antibody or fragment thereof that specifically binds human Fc protein, protein A or protein G, or a combination thereof.

11. The method of any of the preceding claims, wherein the solid support is a bead having a mean diameter at least 2×, 5×, 10×, 50×, 100×, 500×, or 1000× that of the nanoparticle.

12. The method of any of the preceding claims, wherein the solid support is a bead having a mean diameter ranging in size between about 1 μm to about 10 μm or between about 6.5 μm to about 10 μm.

13. The method of any of claims 7-11, wherein the solid support is a bead having a mean diameter of about 3 μm or about 6.5 μm.

14. The method of any of the preceding claims, wherein the fluidic device comprises or consists of a microfluidic chip or sequestration pen.

15. The method of any of the preceding claims, wherein a single clone of cells are seeded into the fluidic device.

16. The method of any of the preceding claims, wherein the optical signal comprises light emission, optical pattern, or light scattering.

17. The method of claim 16, wherein the light emission comprises fluorescence emission.

18. The method of any of the preceding claims, wherein the change in optical signal is detected using an emission filter.

19. The method of any of the preceding claims, wherein the change in optical signal is detected using fluorescence scan.

20. The method of claim 19, wherein the solid support has an average of 6.5-10 μm, such as about 6.5 μm.

21. A system comprising:
a fluidic device;
nanoparticles coated with a ligand for an antibody protein product; and
solid supports coated with a ligand for the antibody protein product,
optionally wherein a mean diameter of the solid supports is greater than that of the nanoparticles.
